(19) United States
(12) Patent Application Publication
(10) Pub. No.: US 2026/0065472 A1
(43) Pub. Date: Mar. 5, 2026

(54) Contrast Agent-Free Virtual Contrast-Enhanced Magnetic Resonance Imaging Apparatus and Data Preprocessing Apparatus and Method thereof

(71) Applicant: THE HONG KONG POLYTECHNIC UNIVERSITY, Hong Kong (CN)

(72) Inventors: Jing CAI, Hong Kong (CN); Wen LI, Hong Kong (CN); Yiming SHI, Hong Kong (CN)

(21) Appl. No.: 19/319,837

(22) Filed: Sep. 5, 2025

(30) Foreign Application Priority Data
Sep. 5, 2024 (CN) 202411238642.5

(51) Int. Cl. G06T 7/00; G06N 3/08; G16H 50/20

(52) U.S. Cl. CPC G06T 7/0012; G06N 3/08; G16H 50/20; G06T 2207/10088; G06T 2207/20081

(57) ABSTRACT

Apparatus and method for contrast agent-free virtual contrast-enhanced magnetic resonance imaging (VCE-MRI) based on a VCE-MRI model of federated learning (FL) are provided. The VCE-MRI model is trained using large-scale, highly heterogeneous multi-center data for data of nasopharyngeal carcinoma (NPC) patients, protecting patient data privacy while guaranteeing high generalization of the model. Apparatus and method for preprocessing VCE-MRI data are also provided. In the preprocessing of the data, a training dataset and/or a test dataset suitable for the FL model from patient data from different medical institutions to be used for model training and/or local verification of the FL model are obtained, respectively. The results of clinical evaluation show that the VCE-MRI model developed in the present application has high generalization and high clinical use value.

### TECHNICAL FIELD

The present application relates to medical image diagnosis technology in general, and more specifically, relates to a contrast agent-free virtual contrast-enhanced magnetic resonance imaging apparatus and a data preprocessing apparatus and method thereof.

### BACKGROUND

Contrast-enhanced magnetic resonance imaging (CE-MRI) can be simply called enhanced magnetic resonance imaging or enhanced MRI, and plays an indispensable role in a variety of clinical scenarios, for example, disease diagnosis, tumor target delineation, and treatment effects analysis, etc. A gadolinium contrast agent (GBCA) is a contrast agent used in magnetic resonance imaging (MRI), which contains the element gadolinium, a metal with very strong paramagnetism. In MRI, the GBCA can help enhance the contrast of image, enabling doctors to see blood vessels and their blood supply sites in a human body more clearly. This GBCA plays a very important role in diagnosing tumors, inflammation and other diseases. The emergence of GBCA has completely improved the practice of MRI, providing valuable lesion information for doctors and greatly improving the accuracy of disease diagnosis. The GBCA is mainly used in enhanced MRI (CE-MRI), and it is estimated that about 40% of MRI scans worldwide involve such imaging every year, with over 30 million doses of GBCA consumed.

CE-MRI requires injection of the GBCA. However, the GBCA poses potential safety risks to patients. Recent research reports show that the use of GBCA is highly related to the incidence of nephrogenic systemic fibrosis (NSF), which is a fatal fibrotic disease that occurs in patients with advanced renal failure. In addition, in recent ten years, researchers have observed that gadolinium is deposited in a variety of tissues of human body after using the GBCA, including brain, liver, kidney, skin and bones, which has nothing to do with the renal function, nevertheless. The mechanism of gadolinium deposition and its long-term effects on patients have not been clearly studied. These safety problems have caused European countries to ban the use of some linear GBCAs since 2017.

In order to avoid using the GBCA, virtual contrast-enhanced MRI (VCE-MRI) based on machine learning (ML) has become a safe alternative to GBCA. There have been alternatives in the field to explore GBCA by ML methods to bypass the use of GBCA. The ML method utilizes a variety of conventional MRI sequences (not using contrast) with complementary information and trains a neural network to learn the mapping of contrast agent-free MRI sequences to CE-MRI in order to synthesize VCE-MRI images that do not use any contrast agent but can achieve effects comparable to real CE-MRI.

However, in VCE-MRI, the generalization of ML models to external data is a major challenge, especially when data privacy regulations prevent cross-institutional sharing of data to train high generalization VCE-MRI models.

Although the applicant's previous research work has achieved relatively good results on internal test data, the generalization of ML models on new data from external institutions still poses challenges. The research shows that the generalization of the model can be improved by including more diverse dataset, especially those data with insufficient representativeness. However, due to the patient privacy protection policies such as the Health Insurance Portability and Accountability Act (HIPAA) in the United States and the General Data Protection Regulation (GDPR) in the European Union, collecting large multi-institutional dataset to train ML models to enhance the generalization of ML models faces major challenges in the medical field.

In order to solve the problem of data privacy, federated learning (FL) has been used to train multi-institutional models without explicitly sharing data. FL is a distributed model training method, which allows local model gradients from medical centers in different locations to be integrated during model training without sharing patient data directly. It thereby ensures patient data privacy while utilizing data from multi-institutional for large-scale model training. FL is particularly important in healthcare, especially for non-representative populations (small amounts of data) and rare diseases, where it may be difficult for individual institutions to collect enough data for robust model training. Compared with other medical imaging methods, MRI data is highly heterogeneous (generally caused by different imaging parameters and imaging devices from different manufacturers), which requires a large number of heterogeneous data (i.e., data acquired from different MRI imaging devices and different imaging parameters) to develop high generalization models.

Chinese patent application CN118115407A discloses a system and corresponding method for magnetic resonance virtual contrast enhancement for tumor target area delineation. The system includes: a data acquisition module for acquiring MRI image data from two or more medical institutions, wherein the MRI image data includes GBCA-free T1w-MRI, T2w-MRI and GBCA enhanced CE-MRI image data; a data preprocessing apparatus, which resamples the MRI image data to unify all images to the same dimension, and normalizes or standardizes the resampled image data; a model training module, basing on a training set selected from a portion of the preprocessed image data, takes T1w-MRI and T2w-MRI image data in the training set as the input of the neural network model, and takes CE-MRI image data in the training set as the learning target of the neural network model to train it; and a virtual image generation module, generating GBCA enhanced VCE-MRI image data through GBCA-free MRI image data from external medical institutions. However, this patent application does not involve developing a high generalization model by utilizing a large number of heterogeneous data acquired from different MRI imaging devices from different medical institutions with different imaging parameters.

### SUMMARY OF THE INVENTION

One purpose of the present disclosure is to provide a safe, accurate and highly generalized contrast-agent alternative method to replace the use of GBCA.

In order to achieve this purpose, the present disclosure provides a contrast agent-free VCE-MRI apparatus for patient privacy protection and a corresponding method, which is based on the VCE-MRI model of FL, and the VCE-MRI model is trained using large-scale, highly heterogeneous multi-center data for data of nasopharyngeal carcinoma (NPC) patients, while protecting the data privacy of patients. The results of clinical evaluation show that the VCE-MRI model developed in the present application has high generalization and high clinical use value.

In one aspect, the present disclosure provides a data preprocessing apparatus for VCE-MRI. The apparatus includes: a receiving module for receiving and storing VCE-MRI patient-data files from different medical institutions, wherein the patient-data files contain different data sequences and different views; a data conversion module for converting the VCE-MRI patient-data file into a three-dimensional MHA file, which contains an image array and basic image information data, wherein the patient-identifying information in the VCE-MRI patient-data file is removed in the conversion; a file selection module for selecting a required three-dimensional MHA file from the three-dimensional MHA file, which includes a required data sequence, wherein the required data sequence includes non-fat-suppressed T1w MRI data, fat-suppressed T2w MRI data, and fat-suppressed CE-MRI data; a resampling module for resampling each data sequence in the required data sequence to generate a resampled three-dimensional MHA file; a registration module for applying image registration to the resampled three-dimensional MHA file and generating a registered three-dimensional MHA file; a slicing module for conducting MHA to NPY conversion on the registered three-dimensional MHA files to convert the registered three-dimensional MHA files into two-dimensional NPY slices; a slice selection module for excluding broken slices from the two-dimensional NPY slices to generate selected NPY slices without broken slices, ensuring the end-to-end mapping of MHA to NPY conversion; a standardization module for applying standardization to process the selected NPY slice to generate a standardized image file; and a dataset module for dividing the data in the standardized image file into a training dataset and a test dataset.

In yet another aspect, the present disclosure provides a data preprocessing method for VCE-MRI. The method includes: receiving and storing VCE-MRI patient-data files from different medical institutions, wherein the patient-data files contain different data sequences and different views; converting a VCE-MRI patient-data file into a three-dimensional MHA file, which contains an image array and basic image information data, wherein the patient-identifying information in the VCE-MRI patient-data file is removed in the conversion; selecting a required three-dimensional MHA file from the three-dimensional MHA file, which includes a required data sequence, wherein the required data sequence includes non-fat-suppressed T1w MRI data, fat-suppressed T2w MRI data, and fat-suppressed CE-MRI data; resampling each data sequence in the required data sequence to generate a resampled three-dimensional MHA file; applying image registration to the resampled three-dimensional MHA file and generating a registered three-dimensional MHA file; conducting MHA to NPY conversion on the registered three-dimensional MHA files to convert the registered three-dimensional MHA files into two-dimensional NPY slices; excluding the broken slices in the two-dimensional NPY slices to generate selected NPY slices without broken slices, ensuring the end-to-end mapping of MHA to NPY conversion; applying standardization to process the selected NPY slice to generate a standardized image file; and dividing the data in the standardized image file into a training dataset and a test dataset.

In yet another aspect, the present disclosure provides a multimodal guided collaborative neural network, MMgSN-Net, for VCE-MRI, wherein in the training process of the multimodal guided collaborative neural network, T1w MRI and T2w MRI data in a training dataset are used as inputs of the MMgSN-Net, and the CE-MRI based on the GBCA is used as a learning target of the MMgSN-Net, wherein, the training dataset is obtained according to the aforementioned data preprocessing apparatus for VCE-MRI.

In another aspect, the present disclosure provides a FL training method for an online federated learning training platform for VCE-MRI synthesis. The method includes the following steps. (i) Global model initialization. At the beginning, the central server of the online FL training platform initializes global model weights using the normal distribution. Then the central server distributes the global model weights w_{g} to the user clients of the cooperative institutions. (ii) local model training. each of the user clients initializes a local model using the received global model weights w_{g}, and conducts a round of training on a local dataset. after a round of training, the ith user client among the user clients obtains the updated local model weights wᵢ and calculates the gradient update uᵢ of the local weights of the ith user client, where uᵢ=wᵢ−w_{g}. Then, the gradient update of the local weights of each of the user clients is uploaded to the central server. (iii) Gradient update aggregation. The central server aggregates the gradient updates of the local models of each of the user clients according to u_{g}=f(u₁, u₂, . . . , uₙ₋₁, uₙ), where f(⋅) represents the aggregation rule, and the aggregation rule is FedProx. Then u_{g} is used to update the global model: w_{g}=w_{g}+ [symbol] ·u_{g}, where [symbol] is the learning rate; wherein, after obtaining the updated global model, the central server sends the new global model weights to the user clients, and the FL training method is continuously iterated until the global model converges; wherein, the local dataset is a training dataset obtained according to the aforementioned data preprocessing apparatus for VCE-MRI.

The present disclosure also provides a computer program product including instructions which, when executed by a computer, cause the computer to perform the aforementioned data preprocessing method for VCE-MRI.

The present disclosure also provides a contrast agent-free VCE-MRI apparatus, which includes an image generation module and the aforementioned data preprocessing apparatus for contrast agent-free VCE-MRI, wherein, the data preprocessing apparatus preprocesses contrast agent-free MRI image data from different medical institutions to generate a training dataset or a test dataset to reduce data deviations from different medical institutions; and the image generation module generates contrast agent-free VCE-MRI image data through a training dataset or a test dataset from a data preprocessing apparatus.

Preferably, the contrast agent-free VCE-MRI apparatus further includes a data acquisition module and a generalization test module; wherein, the data acquisition module acquires MRI image data from two or more medical institutions, and the MRI image data includes contrast agent-free longitudinal relaxation time-weighted magnetic resonance imaging T1w-MRI image data, transverse relaxation time-weighted magnetic resonance imaging T2w-MRI image data and CE-MRI image data enhanced by the contrast agent; the data acquired by the data acquisition module is input into the receiving module of the data preprocessing apparatus for data preprocessing; the generalization test module improves the generalization of the VCE-MRI apparatus by collecting real cancer patient MRI data with scanning parameters of different medical institutions for training, while narrowing the differences between different test dataset and training data.

Therefore, the present disclosure developed a VCE-MRI model based on FL to replace the use of GBCA, compared to existing methods, the model of the present disclosure is trained using large-scale, highly heterogeneous NPC data, which protects the data privacy of patients while guaranteeing the high generalization of the model.

The solution of the present disclosure and the aforementioned CN118115407A belong to different inventive concepts and have different technical points. CN118115407A mainly matches the distribution of internal and external dataset by designing an image distribution matching algorithm, thereby achieving improvement of the performance of neural network model on external dataset without training models, while the solution of the present disclosure focuses on using richer data to train a ML model with higher generalization, which is essentially different from the technical solution in CN118115407A.

The above-mentioned summary of the present disclosure as well as the following detailed description of exemplary embodiments of the present disclosure will be better understood by reading in conjunction with the accompanying drawings. For the purpose of illustrating the present disclosure, illustrative embodiments of the present disclosure are shown in the drawings. However, it should be understood that the present disclosure is not limited to the precise arrangements and instrumentalities shown in the drawings.

### BRIEF DESCRIPTION OF THE DRAWINGS

FIG. 1 is a data preprocessing apparatus according to an exemplary implementation of the present disclosure.

FIGS. 2-5 are schematic screenshots of an online FL training platform according to an exemplary implementation of the present disclosure, wherein:

FIG. 2 is the main page of the online FL training platform;

FIG. 3 is a control panel of the online FL training platform;

FIG. 4 is a real-time training monitoring page of the online FL training platform; and

FIG. 5 is a training page of the user client of the online FL training platform.

FIG. 6 is a schematic diagram of a FL process according to an exemplary implementation of the present disclosure.

FIG. 7 is a schematic structural diagram of the online FL training platform according to an exemplary implementation of the present disclosure.

FIG. 8 is a VCE-MRI apparatus including the data preprocessing apparatus shown in FIG. 1 according to an exemplary implementation of the present disclosure.

### DETAILED DESCRIPTION OF THE INVENTION

The technical solution of the present disclosure is described below through specific embodiments with reference to the accompanying drawings.

The apparatus and method of the present disclosure relates to VCE-MRI technology, and the specific embodiment described below synthesizes VCE-MRI data for NPC patients by applying FL technology. The VCE-MRI technology in the present invention can be used for delineating the target area of radiotherapy for a variety of cancers, for example, NPC, liver cancer, breast cancer and brain cancer, etc.

NPC is a rare disease worldwide, ranking 22nd in incidence among all cancer types, and is mainly found in Southeast Asia, especially southern China. In view of the scarcity of NPC cases worldwide and the strict data privacy policies, it is extremely difficult to collect large-scale and diverse heterogeneous data for centralized training of VCE-MRI technology.

By utilizing FL, the VCE-MRI apparatus and method of the present disclosure can learn by utilizing multi-center heterogeneous data and obtain sufficient knowledge from diverse dataset. By utilizing FL, the VCE-MRI apparatus and method of the present disclosure used unprecedented multi-center heterogeneous data to train the model, including 2187 NPC patients from different institutions, utilizing 92 MRI scanning devices, covering 34 types of MRI devices. This is the biggest difference between the research work of the present disclosure and the previous work in this field.

One of the purposes of the VCE-MRI apparatus and method of the present disclosure is to solve the following practical problem: the generalization of the VCE-MRI model. The existing high-precision but low-generalization VCE-MRI models are only suitable for use within a specific hospital and are not universal, which greatly hinders widespread application of the VCE-MRI technology in clinics. In the research work of the present disclosure, in order to verify the reliability of the synthesized VCE-MRI of the developed FL model, the present disclosure further conducted a clinical evaluation of the synthesized VCE-MRI, which was completed by ten clinicians from eight hospitals, including seven radiologists and three oncologists. Based on the clinical evaluation, in general, the present disclosure provides a secure, accurate and highly generalized contrast agent alternative method to replace the use of GBCA. The model of the present disclosure is trained using large-scale, highly heterogeneous NPC data, while protecting the data privacy of patients.

With reference to the accompanying drawings, the main technical details of the present disclosure are explained from the following three aspects: data, neural network, and FL training.

### 1. Data Preprocessing

The data involved in the present disclosure are all patients with NPC confirmed by biopsy. Each case contains three kinds of magnetic resonance sequence images: non-fat-suppressed T1-weighted (T1w) MRI data, fat-suppressed T2-weighted (T2w) MRI data, and fat-suppressed CE-MRI data based on the GBCA. The present disclosure used multi-center heterogeneous data to train the model. MRI images are acquired from different manufacturers and models of MRI imaging device, including Siemens (Skyra, Prisma, Aera, etc.), Philips (Signa Excite, Signa Pioneer, Optima MR360, etc.), GE (Ingenia, Achieva, Intera, etc.), and United Imaging (uMR 780 and uMR 790). The magnetic field intensity of the scanner is 1.5 T and 3 T.

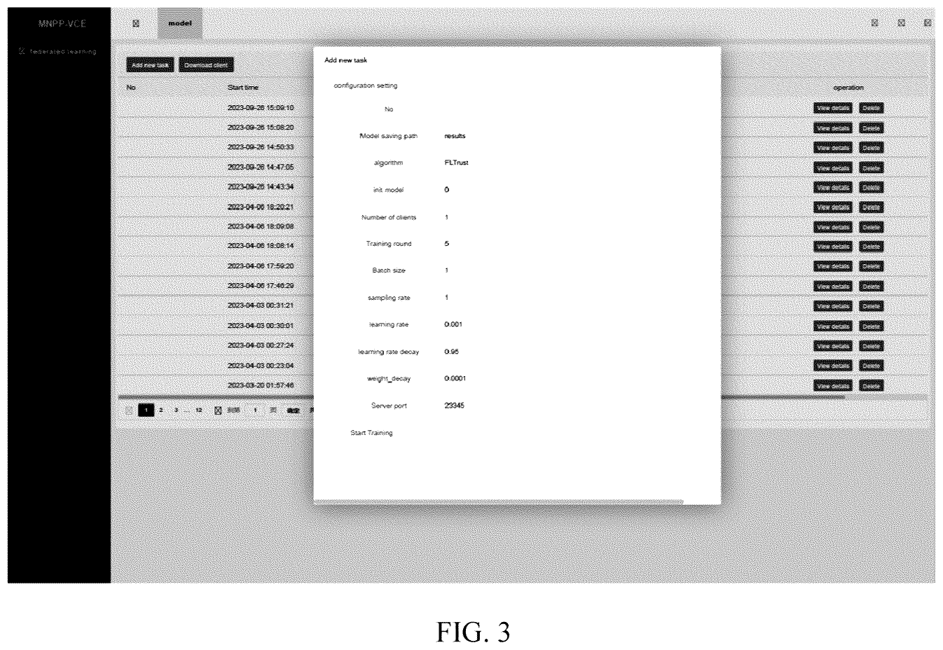

The present disclosure used 2061 patients for training the FL model of the present disclosure (which will be described in detail below), and the remaining 126 patients were used for external verification to evaluate the generalization ability of the FL model. These external verification data did not participate in the training of the FL model. The data of these 126 cases include 45 different MRI imaging devices, of which 23 devices have not been used in the training process (for different device models). Therefore, this external verification set is highly representative, which can more realistically reflect the performance of the FL model of the present disclosure on MRI data scanned by different devices under real conditions. In each central data (for example, the central data in positions 1-14 shown in FIG. 3) participating in the FL model training of the present disclosure, patient cases are randomly divided into a ratio of 4:1 for model training and local verification.

Data preprocessing: Due to the sensitivity of patient data, patient data cannot be taken out of hospitals or medical institutions, so it is necessary to preprocess patient data for further use or analysis. Data preprocessing is done by the staff of various medical institutions. The quality of the training data is very critical to the training of the FL model of the present application, which will directly affect the prediction effects of the FL model of the present application. Considering that different operators may have different data preprocessing methods, the present disclosure provides a data preprocessing method and a corresponding apparatus for VCE-MRI to obtain a training dataset and/or a test dataset suitable for the FL model from patient data from different medical institutions, to be used for model training and/or local verification of the FL model of the present application, respectively.

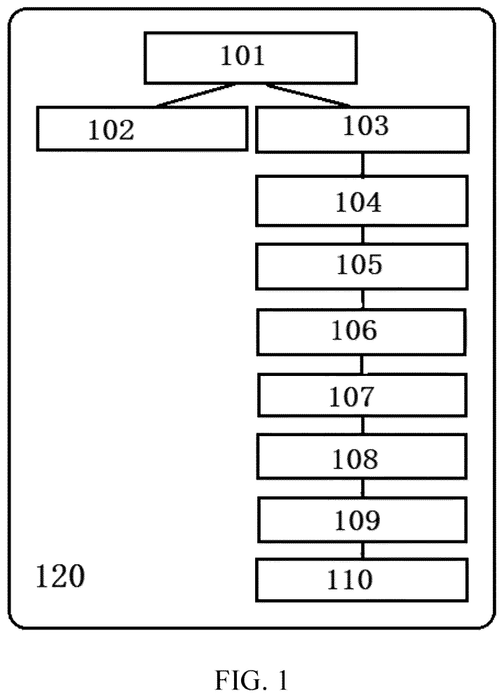

As shown in the data preprocessing apparatus 120 in FIG. 1, the data preprocessing method and corresponding apparatus of the present disclosure establish a set of standard data preprocessing flow (HDP), which is suitable for VCE-MRI to guarantee the consistency of post-processed data and reduce data deviations caused by different data preprocessing methods. FIG. 1 is a schematic diagram of the processing flow of a data preprocessing apparatus 120 of a FL model of the present disclosure, including the following steps.

### 1.1. Data Type Conversion and Anonymization

As shown in FIG. 1, in the receiving module 101, VCE-MRI patient-data files from different medical institutions are received and stored on a local workstation. The patient-data files are usually in DICOM (Digital Imaging and Communications in Medicine) format, which contains different data sequences and different views. The data sequences include, for example, T1w, T2w, CE-MRI, DWI, ADC, STIR, etc. The different views include, for example, Axial, Coronal, Sagottal. For the convenience of analysis. In the data conversion module 103, each data sequence is converted into a three-dimensional MHA file, which contains an image array (one three-dimensional volume) and basic image information data, such as voxel size, spacing, origin and direction. Converting data from DICOM to MHA helps eliminate patient-identifying information in the metadata. MHA is a storage format for volume data, consisting of a header describing the data and the data, and is generally used for medical images.

In the anonymization module 102, patient-identifying information in a patient-data file, for example, a DICOM format, is removed. The patient-identifying information can be stored in another file (for example, Excel file), which can be called header information, including the patient's demographic information (age, gender) and imaging protocol, for example, the manufacturer and model of the MRI imaging device, magnetic field intensity, echo time, repetition time, flip angle, slice thickness, etc. The header information file is private and will be securely saved in a local machine of the corresponding medical institution, which can be used for statistics of patient demographic information, for example. The VCE-MRI apparatus and method of the present disclosure may not use the header information file and the patient-identifying information therein to protect the privacy information of the patient.

After the conversion of the data type of the patient data from DICOM to MHA is completed, in the file selection module 104, the image file of the transverse view of the required data sequence is chosen to generate the required three-dimensional MHA file, which includes the required data sequence (i.e. non-fat suppressed T1w MRI, fat suppressed T2w MRI, fat suppressed CE-MRI). The choice can be manually chosen by medical physicians or radiologists of various medical institutions to ensure accurate selection of the required three-dimensional MHA files, or automatically selected by the file selection module.

### 1.2. Resampling and Registration

After the required MRI data sequence is selected in the required three-dimensional MHA file, in the resampling module 105, resample each data sequence in the required MRI data sequence to a suitable size, for example, 256*256, to generate a resampled three-dimensional MHA file. Then, in the registration module 106, the problem of potential sequence mismatch caused by a respiratory motion is solved by using, for example, rigid registration. Image registration generally includes both rigid registration and non-rigid registration. Rigid registration mainly solves the problem of simple overall image movement (such as translation, rotation, etc.); non-rigid registration mainly solves the problem of flexible transformation of image, which allows the corresponding position relationship between any two pixels to change during the transformation process. The registration module of the present disclosure adopts rigid registration, wherein T1w MRI data is adopted as a reference image and T2w MRI and CE-MRI data are adopted as moving images to generate a registered three-dimensional MHA file 106.

After the rigid registration, in the slicing module 107, the registered three-dimensional MHA file is converted from MHA to NPY for converting the registered three-dimensional MHA files into two-dimensional NPY slices. The NPY file is a NumPy array file created by Python software package with the NumPy library installed. It contains an arrangement that is saved in a NumPy(NPY) file format. Since some slices may lose information due to rotation and become broken slices during the rigid registration process, the slice selection module 108 can select the two-dimensional NPY slices obtained in the slicing module 107 to exclude broken slices to generate selected NPY slices without broken slices, thereby ensuring accurate end-to-end mapping of MHA to NPY conversion. Medical physicists or radiologists of various medical institutions can manually choose two-dimensional NPY slices in the slice selection module 108 to exclude the broken slices; or the slice selection module can automatically select to automatically exclude the broken slices.

### 1.3. Data Standardization

After excluding the broken slices with lost information, in the standardization module 109, standardization is applied to solve the deviation of the pixel intensity distribution of the MRI images between different medical institutions, and a standardized image file is generated. According to past experience, the distribution deviation of image pixel intensity between medical institutions or patients significantly affects the generalization ability of multi-institutional models. In order to alleviate this problem, Z-score normalization based on a single patient is adopted to ensure that the average value of the MRI data of each patient in the VCE-MRI patient-data file is 0 and the standard deviation is 1. Z-score normalization is also called standard deviation normalization. By subtracting the average value from each data and then dividing it by the standard deviation, each data is converted into a standard normal distribution interval with an average value of 0 and a standard deviation of 1. The applicant's research shows that using Z-Score normalization is effective in reducing the deviation of image pixel intensity distribution of MRI data.

### 1.4. Training/Test Dataset

As described above, for each central data participating in the FL model training of the present application (for example, the central data in each task named according to the start time shown in FIG. 3), in the dataset module 110, the data in the standardized image 109 obtained from each patient case according to the above-mentioned processing is randomly divided according to the ratio of, for example, 4:1 to generate a training dataset and a test dataset, which are used for model training and local verification of the FL model of the present application, respectively.

### 2. Neural Network

The neural network adopted in the present disclosure is a multimodal guided collaborative neural network (MMgSN-Net), which is a neural network previously developed by the present applicant and its performance has been confirmed in many studies. The training model of the MMgSN-Net can refer to the documents published by the inventor team of the present invention: Li W, Xiao H, Li T et al. “Virtual Contrast-enhanced Magnetic Resonance Images Synthesis for Patients with Nasopharyngeal Carcinoma using Multimodality-guided Synergistic Neural Network”, Int. J. Radiat. Oncol. 2021; 112 (4): 1033-1044, the entire contents of which are incorporated herein by reference.

During the training process of the MMgSN-Net, the T1w MRI and T2w MRI data in the aforementioned training dataset are used as the input of the MMgSN-Net, while the CE-MRI based on the GBCA is used as the learning target of the MMgSN-Net. The learning rate used by MMgSN-Net is 0.001 and optimized using the Adam (Adaptive Moment Estimation) optimizer. In order to handle the negative values due to Z-Score normalization, the LeakyReLU activation function is used after each convolutional layer to prevent negative values from being truncated.

### 3. FL Training

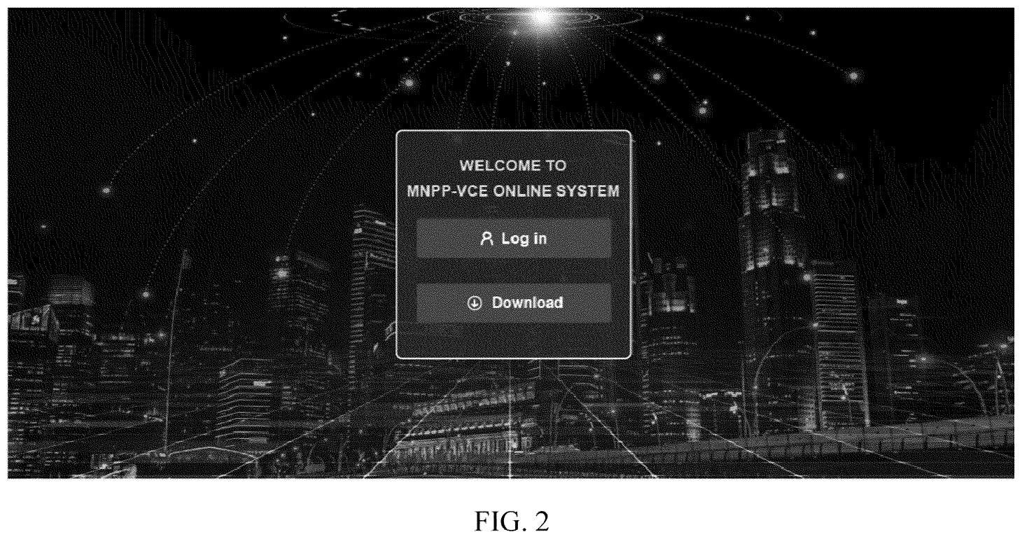

For the training of the FL global model of the MMgSN-Net, the online FL training platform developed for the present disclosure is used, as shown in FIGS. 2-5, and the training process is protected by the firewall of Hong Kong Polytechnic University (PolyU) in Hong Kong, China. The FL training process is shown in FIG. 6.

FIGS. 2-5 are schematic diagrams of an online FL training platform according to an embodiment of the present disclosure.

FIG. 2 is the main page of the online federated learning training platform: the administrator can enter the control panel by clicking the “Log in” button. cooperative institutions can download the user clients of the MMgSN-Net to their local workstations by clicking the “Download”button.

FIG. 3 is the control panel of the online FL training platform: Clicking the “Add new task” button to open the configuration setting menu, and the administrator can input training parameters. After setting the FL training parameters, click the “Start Training” button to start a new FL training task.

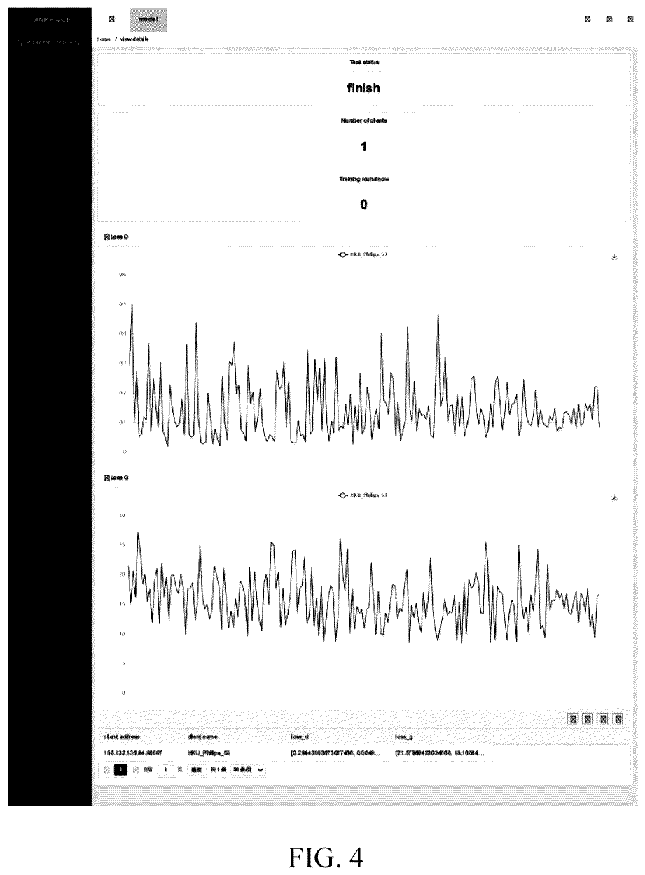

FIG. 4 is the real-time training monitoring page of the online FL training platform: by clicking the “View details” button of the new training task in FIG. 3, the administrator can monitor the real-time changes of the Loss function (Loss) when each cooperative institution trains the model during the training process.

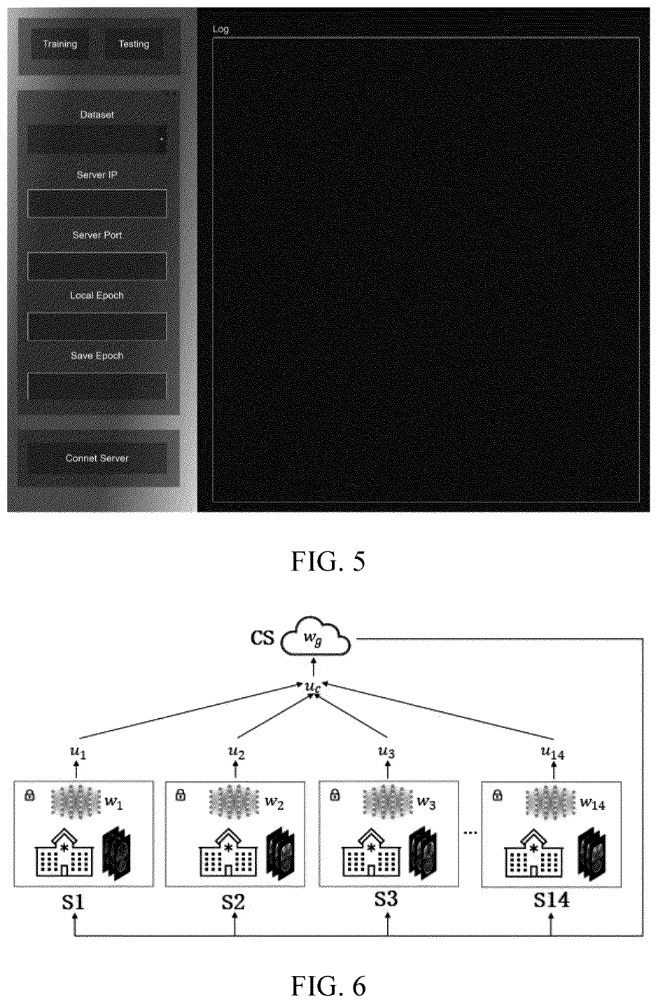

FIG. 5 is the training page of the user client of the online FL training platform: after configuring the local training parameters and clicking the “Connect Server” button at the bottom of the left column in FIG. 5, the local model is connected to the server, and the FL model will automatically start training after all local clients are connected.

FIG. 6 is a schematic diagram of a FL training method according to an embodiment of the present disclosure. As shown in FIG. 6, after all the cooperating medical institutions are securely connected to the central server CS located at PolyU, the training of the FL global model of the MMgSN-Net of the present disclosure automatically starts. At the beginning of the training, the central server CS initializes the weights of the neural network using a normal distribution and then passes the initialized weights to the local models of each cooperative institution. The training process of the FL global model of the present disclosure is as described in FIG. 6. The FL global model of the MMgSN-Net is not a training platform, but a model obtained by the training method described in FIG. 6 utilizing the training platform.

As shown in FIG. 6, after receiving the initialized weights from the central server CS, each cooperative institution S₁, S₂, S₃, . . . , S₁₄ instantiates the same neural network using the received weights W₁, W₂, W₃, . . . , W₁₄, and conducts a round of the local training using their respective local data. After the cooperative institutions complete a round of the local training, the trained weight gradient updates u₁, u₂, u₃, . . . , u₁₄ will be uploaded to the central server CS for aggregation u_{C}. Once the central server CS receives the weight gradient updates of all the cooperative institutions, it will aggregate these weight gradient updates and send the current global model back to each cooperative institution for the next round of training. This process is called “communication round”and represents one iteration of federated training.

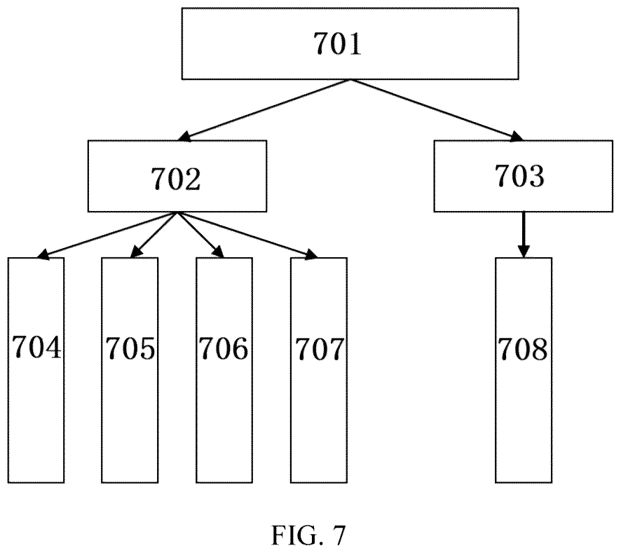

FIG. 7 is a schematic diagram of the structure of the online FL training platform according to an exemplary implementation of the present disclosure. As shown in FIG. 7, the structure of the online FL training platform 701 of the present disclosure includes a central server CS 702 and a user client 703. The central server CS 702 mainly includes four modules, they are an account login module 703 (as shown in FIG. 2), a client download module 704 (see “Download client” shown in FIG. 2), training task management 705 (see “Add new task”→“configuration setting” shown in FIG. 3) and training progress monitoring module 706 (see “view details” shown in FIG. 4), respectively, while user client 703 includes model training module (see “Training” shown in FIG. 5). The online federated learning training platform 701 shown in FIG. 7 of the present application can be independent of the data preprocessing apparatus shown in FIG. 1. The image data acquired from hospitals or medical institutions are preprocessed by the data preprocessing apparatus shown in FIG. 1, and then put on the FL platform shown in FIG. 7 for training.

The gradient update aggregation algorithm adopted in the present disclosure is FedProx, which is superior to FedAvg algorithm (the most classic FL algorithm at present) when it involves the difference of device used for local model training and the heterogeneity of training data between cooperative institutions. In order to ensure that the global model converges well, a total of 100 communication rounds of training are conducted.

As shown in FIG. 6, the FL includes three main steps: the first step: global model initialization, the second step: local model training, and the third step: gradient update aggregation.
Step 1: At the beginning, the central server CS initializes the global model weights using a normal distribution. Then, the central server CS distributes the global model weights (w_{g}) to the user clients S1, S2, S3, . . . , S14 of the cooperative institutions.
Step 2: Each user client S1, S2, S3, . . . , S14 initializes the local model using the received global model weights (w_{g}) and conducts a round of training on the local dataset.

After a round of training, the ith client obtains the updated local model weights (wᵢ) and calculates the gradient update (uᵢ) of the local weights, where uᵢ=wᵢ−w_{g}. Then, the gradient update uᵢ of the local weights is uploaded to the central server CS.
Step 3: The central server aggregates the gradient updates (uᵢ) of the local model according to u_{g}=f(u₁, u₂, . . . , uₙ₋₁, uₙ), where f(⋅) represents the aggregation rule, and FedProx is used in the present disclosure. Then use u_{g} update global model: w_{g}=w_{g}+ [symbol] ·u_{g}, where [symbol] is the learning rate. After obtaining the updated global model, the central server CS sends the new global model weights to the user clients S1, S2, S3, . . . S14 of the cooperative institutions, and this iterative process continues until the global model converges.

The aforementioned data preprocessing method, multimodal guided collaborative neural network MMgSN-Net or online federated learning training platform can all be implemented as computer program products, and executing the data preprocessing method, implementing the MMgSN-Net or online federated learning training platform by instructions in the computer program product.

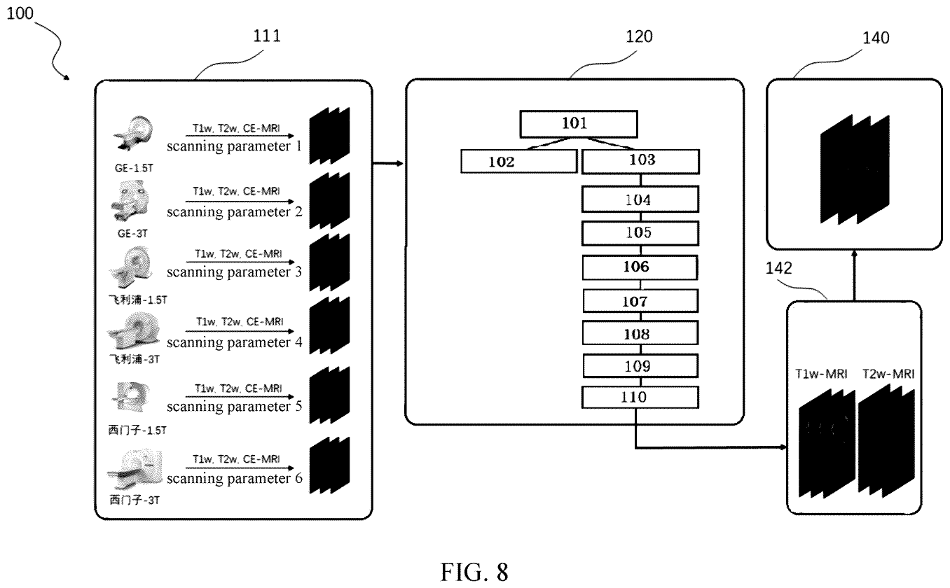

FIG. 8 is a VCE-MRI apparatus 100 according to an exemplary implementation of the present disclosure, which includes the data preprocessing apparatus 120 shown in FIG. 1.

The applicant's prior application CN118115407A discloses a system and method for magnetic resonance virtual contrast enhancement for tumor target area delineation, and the entire disclosure of the prior application is incorporated herein by reference. The VCE-MRI apparatus of the present disclosure basically corresponds to the magnetic resonance virtual contrast enhancement system for tumor target area delineation of the prior application, but adopts the data preprocessing apparatus 120 shown in FIG. 1 of the present disclosure to replace the preprocessing module in the system of the prior application to improve the preprocessing of VCE-MRI patient-data files from different medical institutions, thereby obtaining better VCE-MRI. The training dataset and the test dataset generated by the dataset module 110 in FIG. 1 of the present disclosure can be inputs to the image generation module 140 in FIG. 8 to generate a magnetic resonance virtual contrast enhanced image.

As shown in FIG. 8, the VCE-MRI apparatus of the present disclosure includes: a data preprocessing apparatus 120 as shown in FIG. 1, which is suitable for VCE-MRI, preprocesses contrast agent-free MRI image data from different medical institutions, generates a training dataset or a test dataset to guarantee the consistency of post-processed data and reduce data deviations from different medical institutions; and an image generation module 140, which can generate VCE-MRI image data through the training dataset or the test dataset from the data preprocessing apparatus 120.

As shown in FIG. 8, the VCE-MRI apparatus of the present disclosure may optionally include a data acquisition module 111 and a generalization test module 142.

The data acquisition module 111 acquires MRI image data from two or more medical institutions, wherein, the MRI image data includes contrast agent-free longitudinal relaxation time-weighted magnetic resonance imaging T1w-MRI image data, transverse relaxation time-weighted magnetic resonance imaging T2w-MRI image data and CE-MRI image data enhanced by contrast agent; the data acquired by the data acquisition module 111 can be input into the receiving module 101 of FIG. 1 for data preprocessing.

In FIG. 8, the data acquisition module 111 acquires MRI images under a variety of scanning conditions from different medical institutions. Scanning conditions include, but are not limited to, magnetic field intensity, RF coil sort, spatial resolution of image, phase encoding step, use of fast scanning sequence, scanning time, TR, TE, NSA and combinations thereof. Scanning conditions include, but are not limited to, magnetic field intensity, RF coil sort, spatial resolution of image, phase encoding step, use of fast scanning sequence, scanning time, TR, TE, NSA and combinations thereof. In some embodiments of the present invention, contrast agent-free T1w-MRI, T2w-MRI and contrast enhanced CE-MRI image data of patients with nasopharyngeal carcinoma are collected from different medical institutions. The imaging devices of these medical institutions vary, including MRI scanning devices from companies such as General Electric, Philips, and Siemens.

The data preprocessing apparatus resamples the MRI image data to unify images with different sizes to the same dimension, and normalizes or standardizes the resampled image data, wherein the normalization or standardization includes normalization or standardization based on the entire image dataset, based on a single image data, or based on a single patient image data.

As described in the above-mentioned prior application, the VCE-MRI apparatus of the present disclosure may further include a model training module. The model training module based on the T1w-MRI and T2w-MRI image data in the training dataset or test dataset generated by the dataset module 110 as the input of the neural network model, and takes CE-MRI image data in the training dataset or test dataset as the learning target of the neural network model to train the neural network model.

As mentioned in the prior application, in order to increase the generalization of the model, the VCE-MRI apparatus of the present disclosure may further include a generalization test module 142 to improve the generalization of the model by collecting more real cancer patient MRI data with scanning parameters of different medical institutions for training, while narrowing the differences between different test dataset and training dataset, which will not be elaborated herein.

Based on the description above, the VCE-MRI apparatus and method of the present disclosure mainly involve the following aspects.

Application level: The work of the present disclosure mainly solves the practical problem faced in the clinical application of VCE-MRI technology: insufficient generalization of the model. At present, no related work other than the present disclosure has been published with the purpose of improving the generalization of the VCE-MRI model. The work of the present disclosure is to improve the generalization of the VCE-MRI model. Through the FL flow described in FIG. 6, the inventor of the present application united a number of medical institutions to train the FL model of the present disclosure, while a single-center model was trained by utilizing local data of each institution that participated FL. The image data of the institution that did not participate in the FL model training is called an external dataset. Both the single-center model and the FL model were tested on the external dataset, with the average absolute error as the evaluation metric. The present disclosure found that on the external dataset, the FL model of the present disclosure improved the average absolute error score by 21.23% as compared to the single-center model. This improvement ratio is based on the average absolute error as the evaluation standard.

Technical level: In previous works, the research team applied the FL technique to the medical field. However, but work of the present disclosure applies the FL technology to VCE-MRI synthesis for the first time so as to enhance generalizability of the model, while solving the problem of patient-data privacy. The work of the present disclosure applies the FL technology to the VCE-MRI synthesis task for the first time. That is to say, utilizing the online FL platform shown in FIGS. 2-5 and applying the FL method shown in FIG. 6, a FL global model can be obtained, which can be utilized for VCE-MRI synthesis.

Data level: Thanks to the FL technology, the work of the present disclosure has united multiple medical institutions, guaranteeing that the data does not leave the hospitals or medical institutions to protect the privacy of patient data while being able to utilize a large number of multi-center NPC patient data (2187 cases) to train the global model. Thus, the trained FL model has a significant improvement in generalization compared to the single-center model. The work of the present disclosure uses NPC data from multiple centers to develop a FL model. So far, no related research work in the prior art used NPC data from so many centers to train a model.

Soft hardware: In order to implement FL, the present disclosure uses the laboratory's internal server to build its own FL platform (MNPP-VCE), which is the training platform shown in FIGS. 2-5.

This platform is located within the Hong Kong Polytechnic University (PolyU) in Hong Kong, China, and is managed by members of the inventor team of the present disclosure. The networking network is protected by the PolyU firewall. The present application independently built a FL training platform to conduct real FL training. Unlike the related work of the present application, most of the published federated learning work processes in the prior art are to split a specific dataset and utilize a workstation to simulate federated learning training.

Compared with the single-center model, the FL model of the present disclosure can be trained by utilizing multi-center data. Thanks to the increased amount of data and the heterogeneity of multi-center data, the trained FL model has higher accuracy and generalization, is more reliable and has a greater clinical value in real clinical uses.

The present disclosure relates to applications of FL in VCE-MRI, especially for synthesizing VCE-MRI images of patients with nasopharyngeal carcinoma. With MMgSN-Net as the network, the model of the present disclosure takes T1w MRI and T2w MRI as inputs and CE-MRI as the ground truth when training. The descriptions of FIGS. 1-8 illustrate the technical flow of developing the FL model for VCE-MRI image synthesis. This flow can be applied to VCE-MRI image synthesis of different types of cancer.

In order to further improve the accuracy and generalization of the model, the present disclosure may collect more patient data in the future, develop deep neural networks with better performance, and use better FL aggregation algorithm to further improve the prediction effects of VCE-MRI. Besides being applied to patients with nasopharyngeal carcinoma, the present invention will further collect data of other cancer patients for model training, such as brain cancer, liver cancer, etc., and apply the apparatus and method of the present disclosure to different cancer species.

The entire set of technical flow of the present disclosure is currently a FL model for nasopharyngeal carcinoma, which has been built. In the future it is only necessary to collect other cancer diseases to easily expand the FL model of the present disclosure to other diseases, such as liver cancer, brain cancer, etc. Subsequent technical development, such as neural networks, FL aggregation algorithms, etc. can also be easily updated based on the current framework to obtain better prediction results.

In order to have a more comprehensive understanding of the concept of the present disclosure, many specific details of the embodiments of the present disclosure have been described above. However, it will be obvious to one of ordinary skill in the art that the inventive concepts within the present disclosure can be practiced without these specific details. In other instances, well-known features have not been described in detail to avoid unnecessarily complicating the present disclosure.

As used herein, any reference to “one embodiment” or “an embodiment” means that a particular element, feature, structure, or characteristic described in connection with the embodiment is included in at least one implementation. The appearances of the phrase “in one embodiment” in various places in the specification are not necessarily all referring to the same embodiment. Furthermore, it can be understood that features of one embodiment can be combined with features of other embodiments, even if not explicitly recited or described as a combination.

What is claimed is:

1. A data preprocessing apparatus for contrast agent-free virtual contrast-enhanced magnetic resonance imaging (VCE-MRI), including:
a receiving module for receiving and storing VCE-MRI patient-data files from different medical institutions, wherein the patient-data files contain different data sequences and different views;
a data conversion module for converting the VCE-MRI patient-data file into a three-dimensional MHA file that contains an image array and basic image information data, wherein patient-identifying information in the VCE-MRI patient-data file is removed in the conversion;
a file selection module for selecting a required three-dimensional MHA file from the three-dimensional MHA file, the required three-dimensional MHA file including a required data sequence, wherein the required data sequence includes non-fat-suppressed T1w MRI data, fat-suppressed T2w MRI data, and fat-suppressed contrast enhanced magnetic resonance imaging (CE-MRI) data;
a resampling module for resampling each data in the required data sequence to generate a resampled three-dimensional MHA file;
a registration module for applying image registration to the resampled three-dimensional MHA file to generate a registered three-dimensional MHA file;
a slicing module for conducting MHA to NPY conversion on the registered three-dimensional MHA file such that the registered three-dimensional MHA file is converted into two-dimensional NPY slices;
a slice selection module for excluding broken slices from the two-dimensional NPY slices to generate selected NPY slices that are free from the broken slices so as to ensure end-to-end mapping of MHA to NPY conversion;
a standardization module for applying standardization to process the selected NPY slices to generate a standardized image file; and
a dataset module for dividing data in the standardized image file into a training dataset and a test dataset.

2. The data preprocessing apparatus according to claim 1, further including an anonymization module for removing the patient-identifying information from the patient-data file and storing the patient-identifying information in a header information file, wherein the header information file is private and is securely saved in a local machine of the corresponding medical institution.

3. The data preprocessing apparatus according to claim 1, wherein the VCE-MRI patient-data file is in DICOM format, and the registration module applies rigid registration to the resampled three-dimensional MHA file with the T1w MRI data being used as a reference image to make the T2w MRI and CE-MRI data as moving images.

4. The data preprocessing apparatus according to claim 1, wherein the standardization is conducted by adopting Z-Score normalization based on a single patient to ensure that MRI data of each patient in the VCE-MRI patient-data file has an average value of 0 and a standard deviation of 1.

5. The data preprocessing apparatus according to claim 1, wherein the dataset module randomly divides the data in the standardized image file into a training dataset and a test dataset according to a ratio of 4:1.

6. A data preprocessing method for contrast agent-free virtual contrast-enhanced magnetic resonance imaging (VCE-MRI), including:
receiving and storing VCE-MRI patient-data files from different medical institutions, wherein the patient-data files contain different data sequences and different views;
converting a VCE-MRI patient-data file into a three-dimensional MHA file that contains an image array and basic image information data, wherein patient-identifying information in the VCE-MRI patient-data file is removed in the conversion;
selecting a required three-dimensional MHA file from the three-dimensional MHA file, the required three-dimensional MHA file including a required data sequence, wherein the required data sequence includes a non-fat-suppressed T1w MRI data, a fat-suppressed T2w MRI data, and a fat-suppressed contrast enhanced magnetic resonance imaging (CE-MRI) data;
resampling each data in the required data sequence to generate a resampled three-dimensional MHA file;
applying image registration to the resampled three-dimensional MHA file to generate a registered three-dimensional MHA file;
conducting MHA to NPY conversion on the registered three-dimensional MHA files such that the registered three-dimensional MHA file is converted into two-dimensional NPY slices;
excluding broken slices in the two-dimensional NPY slices to generate selected NPY slices that are free from the broken slices so as to ensure end-to-end mapping of MHA to NPY conversion;
applying standardization to process the selected NPY slice to generate a standardized image file; and
dividing data in the standardized image file into a training dataset and a test dataset.

7. The data preprocessing method according to claim 6, further including removing the patient-identifying information from the patient-data file and storing the patient-identifying information in a header information file, wherein the header information file is private and is securely saved in a local machine of the corresponding medical institution.

8. The data preprocessing method according to claim 6, wherein the VCE-MRI patient-data file is in DICOM format, and the image registration is to apply rigid registration to the resampled three-dimensional MHA file with the T1w MRI data being used as a reference image to make the T2w MRI and CE-MRI data as moving images.

9. The data preprocessing method according to claim 6, wherein the standardization is conducted by adopting Z-Score normalization based on a single patient to ensure that the MRI data of each patient in the VCE-MRI patient-data file has an average value of 0 and a standard deviation of 1.

10. The data preprocessing method according to claim 6, wherein the data in the standardized image file is randomly divided into a training dataset and a test dataset according to a ratio of 4:1.

11. A multimodal guided collaborative neural network (MMgSN-Net) for contrast agent-free virtual contrast-enhanced magnetic resonance imaging (VCE-MRI), wherein in a training process of the MMgSN-Net, T1w MRI and T2w MRI data in a training dataset are used as inputs of the MMgSN-Net, and CE-MRI data based on the gadolinium contrast agent (GBCA) is used as a learning target of the MMgSN-Net,
wherein the training dataset is obtained by the data preprocessing apparatus for VCE-MRI according to claim 1.

12. The MMgSN-Net according to claim 11, wherein the learning rate used is 0.001 and optimized using the Adam optimizer; wherein, in order to handle negative values generated by Z-Score normalization, the LeakyReLU activation function is used after each convolution layer to prevent the negative values from being truncated.

13. A federated learning (FL) training method for an online FL training platform for contrast agent-free virtual contrast-enhanced magnetic resonance imaging (VCE-MRI) synthesis, the method including the following steps:
(i) global model initialization: at the beginning, a central server of an online FL training platform initializes global model weights using a normal distribution; then, the central server distributes the global model weights w_{g} to user clients of cooperative institutions;
(ii) local model training: each of the user clients initializes a local model using the received global model weights w_{g}, and conducts a round of training on a local dataset; after a round of training, the ith user client among the user clients obtains the updated local model weights wᵢ and calculates a gradient update uᵢ of the local weights of the ith user client, where uᵢ=wᵢ−w_{g}; then, the gradient update of the local weights of each of the user clients is uploaded to the central server; and
(iii) gradient update aggregation: the central server aggregates respective gradient updates of the local models of each of the user clients according to u_{g}=f(u₁, u₂, . . . , uₙ₋₁, uₙ) where f(⋅) represents the aggregation rule, and the aggregation rule is FedProx; then uses u_{g} to update the global model: w_{g}=w_{g}+ [symbol] ·u_{g}, where [symbol] is the learning rate; wherein, after obtaining the updated global model, the central server sends the new global model weights to the user clients, and the FL training method is continuously iterated until the global model converges;
wherein the local dataset is a training dataset obtained by the data preprocessing apparatus for virtual enhanced magnetic resonance imaging according to claim 1.

14. A computer program product including instructions which, when executed by a computer, cause the computer to perform the data preprocessing method for contrast agent-free virtual contrast-enhanced magnetic resonance imaging (VCE-MRI) according to claim 6.

15. A contrast agent-free virtual contrast-enhanced magnetic resonance imaging (VCE-MRI) apparatus including an image generation module and the data preprocessing apparatus for VCE-MRI according to claim 1, wherein:
the data preprocessing apparatus preprocesses contrast agent-free MRI image data from different medical institutions to generate a training dataset or a test dataset to reduce data deviations from different medical institutions; and
the image generation module generates VCE-MRI image data through the training dataset or the test dataset from the data preprocessing apparatus.

16. The contrast agent-free virtual contrast-enhanced magnetic resonance imaging (VCE-MRI) apparatus according to claim 15, further including a data acquisition module and a generalization test module, wherein:
the data acquisition module acquires magnetic resonance imaging (MRI) image data from two or more medical institutions, wherein the MRI image data includes contrast agent-free longitudinal relaxation time-weighted magnetic resonance imaging (T1w-MRI) image data, transverse relaxation time-weighted magnetic resonance imaging (T2w-MRI) image data and contrast enhanced magnetic resonance imaging (CE-MRI) image data enhanced by contrast agent;
the data acquired by the data acquisition module is input into the receiving module of the data preprocessing apparatus for data preprocessing; and
the generalization test module improves the generalization of the VCE-MRI apparatus by collecting real cancer patient MRI data with scanning parameters of different medical institutions for training while narrowing differences between different test dataset and training data.
